(19) United States
(12) Patent Application Publication
(10) Pub. No.: US 2014/0277496 A1
(43) Pub. Date: Sep. 18, 2014

(54) EXPANDABLE DEVICE FOR IMPLANTATION IN BODY SPACE

(71) Applicant: Joshua Ammerman, Washington, DC (US)

(72) Inventor: Joshua Ammerman, Washington, DC (US)

(21) Appl. No.: 14/165,094

(22) Filed: Jan. 27, 2014

Related U.S. Application Data
(60) Provisional application No. 61/788,042, filed on Mar. 15, 2013.

(51) Int. Cl. A61F 2/44; A61F 2/46

(52) U.S. Cl. CPC A61F 2/442; A61F 2/4611
USPC 623/17.16

(57) ABSTRACT

An expandable device for implantation in a body space, including an outer frame, and an inner frame slidably engaged with the outer frame. The inner frame includes a tapered wall surface. A ratchet is provided for maintaining an expansion state of the device after moving the inner frame relatively away from the outer frame so as to increase an overall size of the device, while the inner frame maintains engagement with the outer frame. The inner frame is configured to expand with respect to the outer frame in a direction perpendicular to a direction of insertion of the device into the body space.

This application is related to, and claims the benefit of priority from, U.S. Patent Application No. 61/788,042, filed on Mar. 15, 2013, the contents of which are incorporated herein by reference in its entirety.

### BACKGROUND

1. Field

The present invention relates to an implantable device. More specifically, the invention is directed to an implantable device configured to be placed anteriorly within a space, and then expand posteriorly within the space to increase the footprint of the device in the space.

2. Related Art

The general use of an implantable device, such as an intervertebral device, is known in the art. Such devices are surgically implanted to facilitate bone grafting in the disc space. However, they have anatomical limitations, they are often complicated to use, and can be costly to manufacture.

One such related device is disclosed in U.S. Pub. No. 2012/0215316 (hereinafter “the '316 publication”). The related device is disclosed as being insertable into the spine via a lateral direction (FIGS. 1A and 1B of the '316 publication), but when inserted laterally, the device described would create expansion from side to side, i.e., in a medial-lateral direction. Thus, the direction of expansion is parallel to the direction of insertion of the device. In another embodiment, the device is described as being insertable into the spine from either an anterior or posterior direction. However, if the device were inserted from the anterior direction, it would expand parallel to the direction of insertion, i.e., anterior-posterior direction. That is, the device is only expandable in the same direction as the direction in which it is inserted. The device is also shown as being insertable from a posterior approach (FIGS. 2A, 2B of the '316 publication), but this embodiment is inoperable from a medical standpoint. It would be anatomically impossible to insert the device from a posterior approach because this would destroy the spinal nerves. Placement via a posterior approach would cause serious nerve injury. Thus, one skilled in the art would never attempt placement via a posterior approach.

### SUMMARY

The present invention was developed to improve the devices of the related art. In particular, the novel device explained herein differs from any previous devices because it utilizes a novel expansion system and it permits coplanar expansion in either an anterior-posterior or medial-lateral direction depending on the route of placement. Moreover, with the present exemplary embodiments, coplanar expansion can be accomplished either unidirectionally (i.e., anterior or posterior) or bidirectionally (i.e., anterior and posterior simultaneously).

According to a first aspect of an exemplary embodiment, an expandable device for implantation in a body space, includes an outer frame, and an inner frame slidably engaged with the outer frame. The inner frame includes a tapered wall surface. A ratchet is provided for maintaining an expansion state of the device after moving the inner frame relatively away from the outer frame so as to increase an overall size of the device, while the inner frame maintains engagement with the outer frame. The inner frame is configured to expand with respect to the outer frame in a direction perpendicular to a direction of insertion of the device into the body space.

The ratchet may include nubs located on the inner frame and tines located on the outer frame. The nubs on the inner frame might engage with the tines on the outer frame, maintaining an expanded state.

According to another aspect of an exemplary embodiment, there is provided an expansion tool that may be insertable into an opening created by the tapered wall surface of the inner frame and a surface of the outer frame, until the desired expansion state of the device is reached.

According to another aspect of an exemplary embodiment, the outer frame and the inner frame may form one or more openings.

According to another aspect of an exemplary embodiment, the one or more openings may be filled with a bone grafting material.

According to another aspect of an exemplary embodiment, the expandable device may be implanted in an interbody space.

According to another aspect of an exemplary embodiment, a second inner frame may slidably engage with the outer frame. The inner frame and second inner frame may move relatively away from each other, so as to increase an overall size of the device, while the inner frame and second inner frame may maintain engagement with the outer frame.

According to another aspect of an exemplary embodiment, a ratchet system for maintaining an expanded position of an expandable device for implantation in a body space includes nubs located on an inner frame of the expandable device and tines located on an outer frame of the expandable device. The nubs may engage with the tines to maintain an expanded state of the expandable device.

The tines may move over the nubs when the inner frame and the outer frame are moved in a first direction relative to each other and the tines and nubs may prevent the inner frame and the outer frame from moving in a second direction relative to each other.

According to another aspect of an exemplary embodiment, the tines may fail when a sufficient force is applied against the inner frame and the outer frame in the second direction.

According to another aspect of an exemplary embodiment, there is provided a method for using an expandable device for implantation in a body space including inserting an expandable device into the body space. The device may have an inner frame and an outer frame that may be slidably engaged with each other, in an unexpanded state, and inserting an expansion tool into a space created by a tapered wall surface on the inner frame. The inner frame and the outer frame may move relatively away from each other in a coplanar plane, perpendicular to a direction of insertion of the expansion tool, as the expansion tool is further inserted into the space, and withdrawing the expansion tool from the space when a desired state of expansion is obtained.

The expandable device may be inserted into an intervertebral disc space.

The expandable device may be inserted from a lateral approach of the intervertebral disc space.

According to another aspect of an exemplary embodiment, the expansion tool may be inserted into a space defined by the tapered wall surface on the inner frame and a tapered wall surface on a second inner frame. Advancing the expansion tool into the space may move the inner frame and the second inner frame relatively away from each other.

The tapered wall surface on the inner frame may be tapered in a first direction and the tapered wall surface on the second inner frame is tapered in a direction opposite the first direction.

### BRIEF DESCRIPTION OF THE DRAWINGS

The above and other objects, features and advantages of the present invention will become more readily apparent from the following detailed description of exemplary embodiments of the invention, taken in conjunction with the accompanying drawings, in which:

FIGS. 1A-1B illustrate a top plan view and a side view of an exemplary embodiment in an unexpanded state;

FIGS. 1C-1D illustrate a top plan view and a cross-sectional view of the exemplary embodiment in an expanded state;

FIGS. 2A and 2B illustrate schematic and cross-sectional views of the exemplary embodiment in the unexpanded state;

FIGS. 2C and 2D illustrate schematic and cross-sectional views of the exemplary embodiment in the expanded state;

FIGS. 3A-3D illustrate cross-sectional views of the exemplary embodiment of the mechanism of attaching the inner and outer frames;

FIGS. 4A-4D illustrate an exemplary embodiment using a tool to expand the device;

FIGS. 5A-5D illustrate schematic and cross-sectional views of another exemplary embodiment in an unexpanded and expanded state;

FIGS. 6A-6C illustrate the method of assembling the inner and outer frames of the device; and

FIGS. 7A-7G illustrate the method of implanting the device in a body space.

### DETAILED DESCRIPTION

In the following detailed description, standard vertebral anatomical terms are employed with their ordinary and customary meanings. An “anterior direction” means toward the front of the patient. A “posterior direction” means toward the back of the patient. A “medial direction” is toward the mid-line, while a “lateral direction” is away from the mid-line. A “lateral approach” is an entry from a side of the spine toward the spine, whereas an “anterior approach” is an entry from a front of the patient toward the spine. A “superior direction” is being toward the head, whereas an “inferior direction” is being away from the head.

During lateral/anterolateral spinal interbody fusion it is desirable to place a graft or mechanical expandable device as posterior as possible within a disc space to maximize foraminal expansion. However, the neural elements typically limit posterior placement of the retractor system, particularly at the lower lumbar levels. The novel expandable device described herein can expand posteriorly so as to permit itself to be placed anteriorly within the disc space and then, after placement in the disc space, expanded posteriorly to increase foraminal height, disc height and also increase the footprint of the device.

In addition, the inventor has developed a similar mechanical expandable device that is able to expand bidirectionally, in both an anterior and posterior direction, which permits maximal fusion surface for bone growth.

Furthermore, if the device is placed via a true anterior or posterior approach, its expansion mechanism is configured to permit medial lateral expansion and create a large “footprint” for spinal fusion.

An intervertebral device (also known as a “graft”), illustrated in the accompanying Figures is configured to create the above described expansion. The device length and height are set to predetermined dimensions, depending on the circumstances surrounding the procedure, and optionally filled with bone graft material prior to implantation. It will be understood that the device will be provided in different “kits,” with the user choosing the device with the appropriate dimensions at the time of implantation. Once implanted, the graft is able to expand posteriorly (i.e., unidirectionally) by pushing a tool through an aperture along a side of the graft which forces a sloped portion of the graft to migrate posteriorly. Nubs and tines disposed on projections and overhangs of inner and outer graft portions, respectively, are spaced apart from each other a predetermined dimension, for example 1 mm increments, to facilitate locking of the graft into position and maintaining expansion in the desired position.

In one aspect of the device, the graft may be expanded under radiographic guidance to “dial-in” the exact size. Then, the empty portion of the graft exposed during expansion may be backfilled with bone graft material to fill the empty portion if desired. Alternatively, the empty space would fill over time by a self-expanding bone graft or substitute.

A bi-directionally expanding graft would follow a similar design to the unidirectionally expanding graft, but with the expansion mechanism disposed in a center of the device. If the surgeon wishes to achieve medial to lateral expansion, the device may be placed via a true anterior or posterior approach and opened as noted above for the unidirectional device.

Each and every device described herein may be constructed of various medical grade metals or a synthetic substance such as poly ether ether ketone or carbon fiber, etc., and thus, the foregoing description is not intended to limit the scope of the device. Each device, or element thereof, may be constructed using more than one of the substances described.

Surgical techniques to implant the device include an open surgical approach or via various cannula, tubes or other minimally invasive retractors or delivery systems, or any other surgical approach.

A specific structural configuration and method of using the device will now be explained in conjunction with the enclosed drawing figures.

As shown in FIGS. 1A-1D, an intervertebral device 100 is expandable after being placed in the disc space. The letter “A” represents the anterior side of the spine while the letter “P” represents the posterior side of the spine. FIGS. 1A and 1C illustrate top plan views, in the unexpanded and expanded conditions, respectively. FIGS. 1B and 1D illustrate a side view of each of the conditions in FIGS. 1A and 1C, respectively.

Turning to FIGS. 2A-2D, the intervertebral device 100 is again shown in the unexpanded and expanded states. An outer frame 110 and an inner frame 120 are movably attached to each other. As shown in FIG. 2B, for example, the inner frame 120 is disposed at least partially inside the outer frame 110 so that the frames 110, 120 overlap with each other when viewed in the plan view. In both the unexpanded and expanded states, the frames 110, 120 overlap with each other.

The outer and inner frames 110, 120 are secured to each other by a tongue and groove engagement, as seen in FIGS. 3A-3D. For example, outer frame 110 comprises side walls 110a, 110b, 110c, 110d and inner frame 120 comprises side walls 120a, 120b, 120c, 120d. The side walls 110b, 110d are flush with the side walls 120b, 120d. End portions of the side walls 110b, 120b, and 110d, 120d engage with each other using a tongue and groove structure, so that the outer side of inner frame side walls 120b, 120d has a rail 180, as shown in FIG. 3C, that fits inside of or engages with a channel 170 formed in the inner side of outer frame side wall 110b, 120b. The end portions are thus slidably engaged with each other, so that the side walls, and hence the frames, are slidably movable with respect to each other.

It is understood that this is but one embodiment of the tongue and groove arrangement. As seen in FIG. 3D, the rail 185 on inner frame side walls 120b, 120d may be T-shaped and may fit inside of or engage with a channel 175 formed in outer frame side wall that is shaped to accept a T-shaped rail. It is further understood that the rails 180, 185 may be contained on the walls of outer frame 110 while the channels 170, 175 may be formed on the walls of inner frame 120.

The device 100 may be pre-assembled, so that the inner frame 120 is already slidably connected to the outer frame 110 by the rails 180 and the grooves 170, or the device 100 may be assembled at the time of use. To assemble the device 100 at the time of use, the rails 180 of the inner frame 120 are placed in the grooves 170 of the outer frame 110 as seen in FIGS. 6A-6C. The small rail tabs 182 shown in FIG. 6A interact with the small groove tabs 172 to prevent the inner frame 120 from becoming disengaged from outer frame 110. The inner frame 120 is tilted in order for the rail tabs 182 to slide under the groove tabs 172 of the outer frame 110 as shown in FIGS. 6B and 6C, similar to placing a dresser drawer into a dresser. It will be understood that the inner frame 120 and outer frame 110 can be formed without the groove and rail tabs 172, 182, using instead the tines 112 and nubs 122 to keep the outer and inner frames 110, 120 engaged.

The inner frame 120 may include a tapered side edge 130 to facilitate expansion as will be described in more detail later. The outer frame 110 may include tines 112 on the outer frame 110, which engage with nubs 122 on the inner frame 120. The structure of the tines 112 and nubs 122 provides the device with the capability to expand with in a ratchet fashion, wherein the inner and outer frames 110, 120, move with respect to each other incrementally in a controlled manner allowing motion in one direction. This allows the operator to expand the device in a steady motion until a desired size is achieved as discussed in more detail below.

Each of the tines 112 and nubs 122 may be spaced apart from each other by a predetermined dimension. For example, the tines and nubs can be spaced apart in 1 mm increments, to enable ratcheting in small increments until a desired expanded state is obtained. The tines 112 are located on an underside of overhangs 190b, 190d on the side walls 110b, 110d, respectively, of outer frame 110, as seen in FIGS. 3A and 3C. Nubs 122 are located on the top of projections 195b, 195d on side walls 120b, 120d, respectively, of inner frame 120. The tines are resiliently flexible, so that as the tines 112 pass over the nubs 122, they bend until the opposing nubs pass under, but the tines and nubs are rigid enough to hold the inner and outer frames in place with respect to each when an expanding force is not present. In addition, the tines are breakable if a sufficient force is applied in a direction opposing the direction of expansion, as will be explained below.

A center point C, C′ is labeled in the drawings for each frame, 110, 120, respectively. The center points C, C′ are imaginary reference points, since the center of the frames are actually open to allow for grafting.

The center points C, C′ are moved relatively away from each other during the act of expansion. As such, a distance between C-C′ is D1 in the unexpanded state, and the distance between C-C′ is D2 in the expanded state, wherein D2>D1. In the embodiment illustrated in FIG. 2A, the distance D1 may be essentially zero, when the centers of the two frames are aligned at their center points. However, in alternative embodiments, the centers of the frames may not be aligned in the unexpanded state, and thus, the distance D1 will be an amount more than zero. This is further discussed in the embodiments depicted by FIGS. 5A-5B.

The expansion is facilitated by a wedge action, and an understanding of the wedge action is facilitated by FIGS. 4A-4D. A tool 150 is inserted laterally into the device (i.e., from a direction in which the device was inserted into the spine), so as to fit within an opening between the tapered wall 130 of inner frame 120 and the side wall of the outer frame 110. A space 160, for example, is defined by the wall 110a and tapered wall 130. The triangular shaped space may have sides a, b, c as illustrated in FIG. 4A, although the present invention is not limited by this specific shape. Other geometry may be utilized depending on how the tool is inserted and the particular dimensions, restrictions, and idiosyncrasies of the patient's disc cavity.

As the expansion tool 150 inserts further into the space 160 from a lateral direction, it pushes the outer frame 120 in the posterior direction (see FIGS. 4B and 4C). That is, the frame is pushed in a direction perpendicular to the direction in which the tool 150 is inserted. The tines 112 and nubs 122 engage with each other, in a ratchet motion, so that as the tool 150 is further inserted, the device expands incrementally with each relative and successive movement of the tines (see also, FIGS. 2B and 2D). The tool 150 may be inserted as far as desired until a desired expansion state is obtained. A typical expansion may be 4-7 mm. After the final expansion configuration is obtained, the tool is withdrawn from the triangular shaped space by the user, the tool is withdrawn entirely from the patient, and the device is fixed in its expanded state by the engagement of the tines 112 and nubs 122 (FIG. 4D).

The expansion tool 150 has a first end 152 that interacts with the device 100 and space 160. In a preferred embodiment, the first end 152 of the expansion tool 150 is square in shape with rounded corners. In this preferred embodiment, a body 154 of expansion tool 150 is rectangular. In addition, the expansion tool 150 can be modified at a second end 156 by the user or at the time of manufacturing. Modifications, for example, might give the user a better grip on the expansion tool 150 or aid in expanding the device 100. In a preferred embodiment, the second end 156 is square in shape. It will be understood by one skilled in the art that the first end 152, the body 154, and the second end of the expansion tool 150 can be any other shape necessary to expand the device 100 in a body space and can be modified in any fashion so as to provide the user with the best possible control over the expansion tool 150.

The tool 150, 250 will typically be made of titanium, however, other materials may be used. In particular, the tool material may be the same as the interface wall (i.e., tapered wall) of the inner frame or it may be formed of a metal alloy.

The maximum and minimum expansion distance of the inner frame 120 with respect to the outer frame 110 may be determined in several ways, including the length and angle of tapered wall 130 or the width of the expansion tool 150. As discussed above, the expansion of the device can be more finely controlled by using the expansion tool 150. In addition, the smaller the angle of the wall 130, the less the inner frame 120 will be expanded as the expansion tool 150 is advanced into the space 160. It will be understood the device 100 will be available in multiple sizes to allow the user to choose the length and angle of tapered wall 130, as well as the size of the expansion tool 150, depending on the circumstances surrounding the insertion.

As noted above, the tines 112 are configured to be flexible enough to bend as force is applied, but resilient enough to return to their original upright position. The tines are slanted as illustrated in the FIGS. 2B and 2D, in a first direction E which facilitates expansion but restricts movement in an opposing second direction E′ after expansion is completed, as shown in FIG. 2D). The particular angle of the tines may be predetermined based on the amount of force desired to expand and retain the device in position.

In some embodiments, the force exerted on the device 100 in an interbody space is in a superior-inferior (i.e., vertical) direction, which is perpendicular to the movement of the inner frame 120 with respect to the outer frame 110. The tines 112 move over the nubs 122 when a force is applied against the inner frame 120 by the expansion tool 150 to move inner frame 120 in a first direction E. Therefore, the tines 112 need only be strong enough to prevent inner frame 120 from moving in a second direction E′ back into outer frame 110. Tines 112 can be relatively weak because there is a minimal force in this coplanar direction. Thus, a user can remove the device 100 from an interbody space by applying a sufficient force against the inner frame 120 in the second direction E′, to break the tines 112 and collapse the inner frame 120 back into the outer frame 110. The device 100 may be removed in the same or different manner than which it was inserted. It will be understood that the “sufficient force” necessary to break tines 112 will be determined by material used and the size of the tines 112.

An alternative embodiment of the expandable device is shown in FIGS. 5A-5B. In this embodiment, the structural configuration is similar to the first embodiment, however, a device 200 is bidirectionally expandable in both the posterior and anterior directions or the medial and lateral directions. The device 200 includes an outer frame 210 similar to the outer frame 110 of the unidirectional device 100. Inner frames 220A, 220B are provided within the outer frame 210 in the initial unexpanded state.

Each of the inner frames 220A, 220B include a tapered wall 230A, 230B, which tapers inwardly from a side wall of the frame towards a center portion as shown in FIG. 5A. A triangular space is created, defined by the tapered walls 230A, 230B, where the tapered walls 230A, 230B are tapered away from each other. An expansion tool 250 is inserted into the space from a lateral direction, similar to the earlier embodiment. However, since two tapered walls are provided, and the tool is inserted proximate or at a center of the device, the device expands bidirectionally, with the tapered wedge action created by both frames 220A, 220B interacting with the tool 250, so that the frames 220A, 220B move in opposite directions away from each other, and in a direction perpendicular to the insertion direction of the tool 250. As previously discussed, the length and angle of tapered walls 230A, 230B can be modified during the manufacturing process.

Tines 212 are provided on an overhang (not shown) of outer frame 210b, 210d, as seen in FIGS. 5C-5D. Also shown in FIGS. 5C-5D are nubs 222 provided on projections (not shown) of inner frames 220b, 220d, respectively. Tines 212 engage with nubs 222 in a ratcheting manner, so that as the tool 250 is further inserted into the triangular shaped space, the tool pushes against the tapered walls 230A, 230B, thereby simultaneously causing the ratcheting of the inner frames 220b, 220d with the outer frame 210b, 210d. Once a desired expansion state is obtained, the tool is withdrawn from the space and the device 200 is in the final expanded state.

Bone grafting material is provided in the open spaces provided by the expanding device. That is, since the device expands in the anterior-posterior direction, there is an increased surface area for bone growth between adjacent vertebrae. This bigger and wider footprint improves the fusion result. For example, a comparison of the top views of the device in the unexpanded and expanded states, for example in FIGS. 4A and 4D, illustrates the enlarged footprint. Since holes or openings are provided within the frames, bone grafting material can be disposed not only within the openings created inside the overlapping frames, but also in the holes/openings that are not a part of the overlap. Also, since the device expands in a coplanar direction only, there is no concern with regard to height restrictions. That is, the device only expands in a coplanar direction, as opposed to other conventional devices which expand vertically to restore height between adjacent vertebrae.

A method of using the devices 100, 200 will now be described. The spinal column is accessed via traditional open, tubular, minimally invasive, laparoscopic or other accepted methods as shown in FIGS. 7A and 7B. Existing disc material is removed in the usual fashion. The desired graft is selected with regards to height, length, closed width, and expanded width. The graft is filled with autologous, allograft, or synthetic bone grafting material. It is impacted into the disc space with various tools. FIG. 7C shown an expansion tool is inserted into the exposed lateral aperture and advanced parallel to the direction of device insertion. The expansion tool will travel between the inner wall of the outer piece (or frame) of the device and the angled or wedge shaped outer wall of the inner piece (or frame) of the device as seen in FIGS. 7D and 7E. This will force the inner piece away from the outer piece in a direction perpendicular from the direction of initial device insertion. As the inner piece moves away from the outer piece the nubs on the inner piece will click incrementally over the tines on the inner surface of the outer piece permitting a controlled and locked expansion. When the desired degree of expansion is achieved, for instance as seen in FIG. 7F, the expansion tool is withdrawn and the gap created between the inner and outer pieces by the expansion is back filled with bone grafting material. The device can then be implanted in a space as seen in FIG. 7G.

The device 100 can be formed in different sizes to fit different interbody spaces. The different sizes can include different lengths of the frame sides 110a-110d, 120a-120d, the angle and length of the tapered wall 130, the distance between the tines 112 and nubs 122, the length of the expansion tool 150, etc. The inner frame 120 and outer frame 110 are formed so the inner frame 120 will be flush with and slidable within the outer frame 110.

It will be understood by one of ordinary skill in the art that the device 100 may be used to create or maintain a space within a body. In particular, the device 100 may be used to separate two elements in a body. In addition, the device 100 can be inserted and expand to provide a space through which medical devices may be implanted, or to perform other medical procedures requiring the creating of a body space or enlarging of a body space.

The present invention provides an expandable device for implantation in a body space, wherein the device is inserted into the space from a direction which is perpendicular to an expansion direction of the device. Thus, if the device is inserted from a lateral approach, a tool is also inserted from a lateral approach, and the device is expanded in a posterior and/or anterior direction. Some of the advantages gained by this structure include the ability to increase the surface area of bone grafting in the posterior area of the spine, without risking damage to the nerves and while using as small an incision as possible.

While the invention has been shown and described with reference to certain preferred embodiments, it will be understood by those skilled in the art that various changes and modifications in form and detail may be made therein without departing from the spirit and scope of the invention, as defined by the appended claims.

What is claimed is:

1. An expandable device for implantation in a body space, comprising:
an outer frame;
an inner frame, slidably engaged with the outer frame, the inner frame comprising a tapered wall surface; and
a ratchet for maintaining an expansion state of the device after moving the inner frame relatively away from the outer frame so as to increase an overall size of the device, while the inner frame maintains engagement with the outer frame,
wherein the inner frame is configured to expand with respect to the outer frame in a direction perpendicular to a direction of insertion of the device into the body space.

2. The expandable device of claim 1, wherein the ratchet includes:
nubs located on the inner frame and tines located on the outer frame,
wherein the nubs on the inner frame engage with the tines on the outer frame, maintaining an expanded state.

3. The expandable device of claim 1, further comprising:
an expansion tool,
wherein the expansion tool is insertable into an opening created by the tapered wall surface of the inner frame and a surface of the outer frame, until the desired expansion state of the device is reached.

4. The expandable device of claim 1, wherein the outer frame and the inner frame form one or more openings.

5. The expandable device of claim 4, wherein the one or more openings are filled with a bone grafting material.

6. The expandable device of claim 1, wherein the expandable device is implanted in an interbody space.

7. The expandable device of claim 1, further comprising:
a second inner frame slidably engaged with the outer frame,
wherein the inner frame and second inner frame are moved relatively away from each other, so as to increase an overall size of the device, while the inner frame and second inner frame maintain engagement with the outer frame.

8. A ratchet system for maintaining an expanded position of an expandable device for implantation in a body space, comprising:
nubs located on an inner frame of the expandable device; and
tines located on an outer frame of the expandable device, wherein the nubs engage with the tines to maintain an expanded state of the expandable device.

9. The ratchet system of claim 8, wherein the tines move over the nubs when the inner frame and the outer frame are moved in a first direction relative to each other, and
the tines and nubs prevent the inner frame and the outer frame from moving in a second direction relative to each other.

10. The ratchet system of claim 8, wherein the tines will fail when a sufficient force is applied against the inner frame and the outer frame in the second direction.

11. A method for using an expandable device for implantation in a body space, comprising:
inserting an expandable device into the body space, which comprises an inner frame and an outer frame that are slidably engaged with each other, in an unexpanded state;
inserting an expansion tool into a space created by a tapered wall surface on the inner frame, wherein the inner frame and the outer frame move relatively away from each other in a coplanar plane, perpendicular to a direction of insertion of the expansion tool, as the expansion tool is further inserted into the space; and
withdrawing the expansion tool from the space when a desired state of expansion is obtained.

12. The method of claim 11, wherein the expandable device is inserted into an intervertebral disc space.

13. The method of claim 12, wherein the expandable device is inserted from a lateral approach of the intervertebral disc space.

14. The method of claim 11, wherein the expansion tool is inserted into a space defined by the tapered wall surface on the inner frame and a tapered wall surface on a second inner frame, wherein advancing the expansion tool into the space moves the inner frame and the second inner frame relatively away from each other.

15. The method of claim 14, wherein the tapered wall surface on the inner frame is tapered in a first direction and the tapered wall surface on the second inner frame is tapered in a direction opposite the first direction.
